(19) United States
(12) Patent Application Publication
(10) Pub. No.: US 2011/0223622 A1
(43) Pub. Date: Sep. 15, 2011

(54) Methods and Kits for Predicting the Onset of Labour

(75) Inventor: Roger Smith, Newcastle (AU)

(73) Assignee: NEWCASTE INNOVATION LIMITED

(21) Appl. No.: 13/055,665

(22) PCT Filed: Jul. 24, 2009
(86) PCT No.: PCT/AU2009/000949

§ 371 Date: Apr. 27, 2011

(30) Foreign Application Priority Data
Jul. 25, 2008 (AU) 2008903832

(51) Int. Cl. G01N 33/74; G01N 21/64

(52) U.S. Cl. 435/7.92; 435/7.1; 436/501

(57) ABSTRACT

The present invention relates to methods and kits useful for predicting the time of onset of labour in a pregnant subject. In particular, the invention relates to methods and kits for predicting the time of onset of labour wherein the levels of at least two hormones, selected from estriol, estradiol and progesterone, are determined and a ratio of said levels is calculated, and wherein the time of onset of labour is predicted by comparison of said ratio with a predetermined ratio. The invention further contemplates methods for preventing preterm delivery of an infant/offspring.

TECHNICAL FIELD

The present invention relates to methods and kits useful for predicting the time of onset of labour in a pregnant subject. In particular, the invention relates to methods and kits useful for predicting the time of onset of preterm labour in a pregnant subject. The invention further contemplates methods for preventing preterm delivery of an infant/offspring.

The invention has been developed primarily for use in pregnant human females and will be described with reference to this application. However, it will be appreciated that the invention is not limited to this particular field and that the invention may be useful in other pregnant mammals.

### BACKGROUND

Any discussion of the prior art throughout the specification should in no way be considered as an admission that such prior art is widely known or forms part of common general knowledge in the field.

The prevalence of preterm labour experienced by pregnant women in developed countries is approximately 10% (Iams J. N. Engl. J. Med. 1998; 338: 54-6). Preterm labour is a major contributor to the morbidity and mortality of neonates and is responsible for 60% of all perinatal deaths (Goffinet et al J Gynecol. Obstet. Biol. Reprod. (Paris) 1997; 26:623-9). Accordingly, the ability to detect an imminent preterm labour would be important, so that if necessary, appropriate medical intervention may be administered to prevent the preterm labour and thus preserve the well-being or survival of the neonate. However, precise determination of an imminent preterm labour is essential as an inaccurate result may lead to either unnecessary treatment that may be detrimental to the pregnancy or, alternatively, may result in an imminent preterm labour going undetected.

Preterm labour may be defined as progressive dilation, shortening and/or thinning of the cervix with consistent contractions resulting in birth before 37 weeks of gestation.

The traditional diagnosis of an impending preterm labour includes either evaluation of one or more of the following conditions: frequency of uterine contractions; membranes status; dilation, shortening or thinning of the cervix; and gestation stage. However, as there has been no significant reduction in preterm births in the past 20 to 30 years, the traditional methods either have limited efficacy or predict the onset of labour at a stage that is too advanced for prophylactic medications such as tocolytic agents or corticosteroids to successfully delay the birth.

More recently a number of markers of impending preterm delivery have been investigated including progesterone, foetal fibronectin, inflammatory factors such as IL-6, and salivary estriol. However, an accurate prediction as to the time of onset of labour remains problematic.

Progesterone has been identified as one of the principal hormones responsible for the maintenance of human pregnancy. In many animals the process of parturition is initiated by a decrease in the systemic level of maternal progesterone (Csapo 1956;) However, although several studies have reported a reduction in preterm birth rates following progesterone supplementation in women at high risk for a singleton preterm birth (Meis et al. 2003; da Fonseca et al. 2003) there does not appear to be a substantial decrease in maternal progesterone plasma concentrations at the onset of labour in human pregnancies (Boroditsky et al 1978; Tulchinsky et al 1972; Mesiano 2001).

On the basis that damage to foetal membranes may be a factor in the onset of labour, a number of studies have proposed that the release of foetal fibronectin into the cervical and vaginal secretions may be indicative of the onset of labour (U.S. Pat. No. 5,650,394). A similar rationale has been used for postulating that the presence of inflammatory products such as IL-6 in cervicovaginal secretions may also be indicative of an impending labour (U.S. Pat. No. 5,5 6,702).

Both the foetal fibronectin and inflammatory factor assays are invasive, requiring samples to be taken from the vaginal canal or cervix. Moreover, the fibronectin test has also been shown to have limited efficacy (58% sensitivity) in determining the onset of a preterm delivery in symptomatic pregnant women before 37 weeks of gestation. (Gibb W 1998 Ann Med 30 235-241).

It has also been reported that salivary estriol levels typically increase several weeks prior to the onset of labour. However, this marker is considered unreliable for predicting the onset of labour. Specifically, the American College of Obstetricians and Gynaecologists does not recommend the use of salivary estriol levels for predicting the onset of labour as it produces a high percentage of false positive results and, as such, could potentially add significant costs and unnecessary interventions to prenatal care. This view is consistent with the increase of estriol levels occurring several weeks prior to the onset of labour. Accordingly, maternal estriol levels appear to be unreliable for prediction of the onset of labour.

It is an object of the present invention to overcome or ameliorate at least one of the disadvantages of the prior art, or to provide a useful alternative.

### SUMMARY OF INVENTION

There is a marked variability of absolute levels of hormones between individual women during pregnancy, often by several orders of magnitude. This makes use of hormone levels as a marker of pregnancy progression problematic. Moreover, to date such levels have been of limited value in identifying subjects who are susceptible to early onset of labour, ie preterm labour. In the present study, hormonal data was viewed in the form of trajectories across gestation and it was surprisingly found that changing ratios in estriol and estradiol levels especially when combined with ratios of progesterone and estrogens are robust characteristics of labour onset. Accordingly, it would appear that target cells may respond to relative changes in hormone concentrations rather than to absolute concentrations.

Serial data on individual pregnancies were used to develop equations that describe the dynamic change in hormonal levels, which allow results for individual women to be effectively compared with each other to find common patterns. This methodology reduces the effect of measurement error for individual samples and assay results and enables comparison between pregnancies where samples are not taken at identical gestational ages and would otherwise need adjustment.

The equations developed on data from early and mid gestation were used to estimate results at labour. It was surprisingly found that the actual levels of a number of hormones measured at labour differed to that of the predicted results. In particular, most measured progesterone results were lower than predicted results, indicating that these trajectories had peaked in the previous month, most measured estriol results were higher than predicted, indicating an estriol surge in the last month in many pregnancies.

It has also surprisingly been found that changes in the ratios of hormone levels may contribute to the onset of labour. In particular, in certain embodiments of the present invention the ratio of estrogens and/or progesterones may be measured to predict the time of onset of labour.

The present invention is based on the principle that changes in the levels and ratios of circulating hormones can produce significant biological effects or responses. Specifically, for example, estriol is an estradiol antagonist at low concentrations but becomes an estrogen agonist as the concentration increases.

The present invention relates, in certain embodiments, to the assessment of the ratio of estriol to estradiol in a pregnant subject as a means for predicting the timing of the onset of labour. The present invention further relates, in certain embodiments, to the assessment of the ratio of estriol to estradiol in combination with the ratio of progesterone to estrogens in a pregnant subject for predicting the timing of the onset of labour. In certain embodiments the method of the invention is useful for predicting a preterm delivery.

In alternate embodiments the method of the invention is useful for predicting imminent delivery at term, that is a normal full-term delivery. Many women require their labour to be induced by administration of, for example, prostaglandins or by mechanical methods such as deliberately rupturing the membranes to produce the onset of labour. Such induction may be carried out for social or convenience reasons but also to produce a delivery if the woman is suffering from a pregnancy related pathology such as pre-eclampsia as this condition is cured by delivery of the baby. Frequently methods for inducing delivery are ineffective if the onset of labour is not already imminent. A failure to induce the onset of labour frequently results in a caesarean section. The method of the invention could be used to identify women who are suitable or not suitable for induction of labour at or after full term. As such, use of the method of the invention could reduce the rate of caesarean sections.
Accordingly, in a first aspect, the present invention provides a method of predicting time of onset of labour in a pregnant subject, said method comprising the steps of:
(a) determining levels of at least two hormones in said pregnant subject;
(b) calculating a ratio using said levels; and
(c) predicting the time of onset of labour in said pregnant subject by comparison of said ratio with a predetermined ratio.

Preferably, the hormones are selected from the group consisting of steroid hormones, peptide hormones and prostaglandins. More preferably, the hormones are selected from the group consisting of estrogens and progesterones. Most preferably, the hormones are selected from the group consisting of estriol, estradiol and progesterone. In a particularly preferred embodiment the hormones are estriol and estradiol. In an alternative embodiment the hormones are estriol and progesterone. Preferably the ratio of progesterone to estriol is in the range of about 4:1 to 0.5 to 1. In particular the ratio of progesterone to estriol is about 4:1, 3.5:1, 3:1, 2:1, 1.5:1, 1:1, or 0.5:1

In yet another embodiment the hormones are estradiol and progesterone. In a further embodiment timing of onset of labour is determined by measuring the ratio of estriol and estradiol combined with the ratio of progesterone to estrogens.

Preferably, an increase in the ratio of estriol to estradiol is indicative of increased susceptibility to onset of preterm labour. More preferably, when the ratio of estriol to estradiol is in the range of about 8:1 to about 12:1 the risk of onset of preterm labour is increased. In particular, when the ratio of estriol to estradiol is about 8:1, 9:1, 10:1, 11:1, or 12:1 the risk of onset of preterm labour is increased.

Preferably, the ratio of progesterone to estriol is in the range of about 4:1 to 0.5 to 1. In particular, when the ratio of progesterone to estriol is about 4:1, 3.5:1, 3:1, 2:1, 1.5:1, 1:1, or 0.5:1 the risk of onset of preterm labour is increased.

Preferably, the ratio of progesterone to estradiol to is in the range of about 14:1 to about 9:1. In particular, when the ratio of progesterone to estradiol to is in the range of about 14:1, 13:1, 12:1, 11:1, 10:1, or 9:1, the risk of onset of preterm labour is increased.

In certain embodiments of the invention, the onset of labour is preterm labour. In alternative embodiments of the invention, the labour is full term labour. The skilled addressee will understand that the methods of the present invention can be used to predict the onset of either preterm or full term labour.

Preferably, the methods of the invention further comprise the step of
(d) providing a diagnosis as to susceptibility to preterm labour.

Preferably, the subject in the methods of the invention is a human.

In one embodiment, levels of the hormones are determined from a blood sample. In an alternative embodiment levels of the hormones are determined from a saliva sample. In a further embodiment levels of the hormones are determined from a cervical or vaginal secretion.

Preferably, the levels of the hormones are determined by an assay that detects the hormones. More preferably, the levels of the hormones are determined by an immunoassay. Most preferably, the levels of the hormones are determined by an assay selected from the group consisting of a competitive immunoassay, an enzyme immunoassay, a ligand assay, an immunoradiomeric assay, a fluoroimmunoassay, an enzyme-linked immunosorbent assay or a radioimmunoassay.

In an alternative embodiment levels of the hormones are determined by measuring nucleic acid molecules encoding said hormones. In one embodiment the nucleic acid is RNA. In another embodiment the nucleic acid is DNA.

In a second aspect, the present invention provides a method of preventing a preterm delivery in a pregnant subject, said method comprising the steps of:
(a) determining susceptibility to onset of preterm labour in said pregnant female subject according to the method of the invention; and
(b) administering treatment to said pregnant female subject such that said preterm delivery is prevented.

Preferably, the treatment comprises the step of administering drug therapy to the subject. More preferably, said drug therapy comprises the step of administering a tocolytic agent or a steroid. Preferably, the tocolytic agent is selected from the group consisting of terbutaline, ritodrine, nifedepine, magnesium sulfate, indmethacin, betamethasone. Preferably, the steroid is progesterone.

Preferably, the levels of the hormones are determined after week 26 of gestation. Preferably, said levels of said hormones are determined between the period of week 26 to week 37 of gestation.

Accordingly, in a third aspect, the present invention provides use of levels of at least two hormones, the levels determined from a sample from a pregnant subject, to calculate a ratio for determining time of onset of labour in said pregnant subject.

Preferably, the hormones are selected from the group consisting of steroid hormones, peptide hormones and prostaglandins. More preferably, said hormones are selected from the group consisting of estrogens and progesterones. Most preferably, the hormones are selected from the group consisting of estriol, estradiol and progesterone. In a particularly prefered embodiment the hormones are estriol and estradiol. In an alternative embodiment the hormones are estriol and progesterone. In yet another embodiment the hormones are progesterone and estradiol.

Preferably, the timing of onset of labour is determined by a ratio of estriol and estradiol combined with a ratio of progesterone to estrogens.

Preferably, an increase in the ratio of estriol to estradiol is indicative of increased risk to onset of labour.

Preferably, a ratio of estriol to estradiol in the range of about 8:1 to about 12:1 is indicative of increased risk to onset of labour.

Preferably, a decrease in the ratio of progesterone to estrogens is indicative of increased risk to onset of labour.

Preferably, a ratio of progesterone to estriol within the range of about 4:1 to about 1:1 is indicative of increased risk to onset of labour.

Preferably, a ratio of progesterone to estradiol in the range of about 14:1 to about 9:1 is indicative of increased risk to onset of labour.

In certain embodiments labour is preterm labour in alternative embodiments labour is full term labour.

Preferably, the subject in the methods of the invention is human.

In one embodiment the levels of the hormones are determined from a blood sample.
In an alternative embodiment the levels of said hormones are determined from a saliva sample.
In yet another alternative embodiment the levels of the hormones are determined from a cervical or vaginal secretion.

Preferably, the levels of said hormones are determined by an assay that detects the hormones.

Preferably, the levels of the hormones are determined by an assay that detects the hormones. More preferably, the levels of the hormones are determined by an immunoassay. Most preferably, the levels of the hormones are determined by an assay selected from the group consisting of a competitive immunoassay, an enzyme immunoassay, a ligand assay, an immunoradiomeric assay, a fluoroimmunoassay, an enzyme-linked immunosorbent assay or a radioimmunoassay.

In an alternative embodiment levels of the hormones are determined by measuring nucleic acid molecules encoding said hormones. In one embodiment the nucleic acid is RNA. In another embodiment the nucleic acid is DNA.

In one embodiment, the levels of the hormones are determined after week 26 of gestation. In an alternative embodiment, the levels of the hormones are determined between the period of week 26 to week 37 of gestation.

In a fourth aspect, the present invention provides a kit when used in a method according to the invention.

Preferably, the kit comprises; sample collection means; and hormone assay means.

The skilled addressee will understand that the invention comprises the embodiments and features disclosed as well as all combinations and/or permeations of the disclosed embodiments and features.

### Definitions

In the context of the present invention, the words “comprise”, “comprising” and the like are to be construed in their inclusive, as opposed to their exclusive, sense, that is in the sense of “including, but not limited to”.

In the context of the present application the term “preterm labour” may be defined as progressive dilation, shortening and/or thinning of the cervix with consistent contractions resulting in birth before about 37 weeks of gestation.

The term “hormone” is well understood in the art and, in the context of the present invention, the term includes all relevant hormones. For example, the term refers to: a hormone from the anterior pituitary gland such as, but not limited to, follicle-stimulating hormone (FSH) or luteinizing hormone; an anterior pituitary-like hormone from the placenta; atrial natriuretic hormone (ANP); a hormone from the stomach, duodenum, pancreas or liver; parathyroid hormone (PTH) or calcitonin; erythropoietin; renin; an interleukin (IL); an oligopeptide hormone; a hormone from the posterior pituitary gland; a hormone from the hypothalamus; angiotensin I or II; an amine hormone (derived from the amino acid tyrosine) such as, but not limited to, a thyroid hormone (T3 and T4), adrenalin, noradrenaline; dopamine (PIH); a steroid hormone such, as but not limited to, a sex hormone from the gonads or placenta such as, but not limited to, corticotrophin-releasing hormone, a progesterone or estrogen including, but not limited to, estriol, estradiol or estrone, an adrenal cortex hormone, 1,25 dihydroxy-Vit D3; a lipid-compound such as, but not limited to, an eicosanoid—prostaglandin, prostacyclin, thromboxane or a leukotriene.

The term “amine hormone” as used in the present application refers specifically to a hormone derived from the amino acid tyrosine.
Throughout the specification, the following abbreviations have been used:
E3=estriol
E2=estradiol
P=progesterone

It will be understood by the person skilled in the art that detection of hormone levels as contemplated in the embodiments of the present invention is not limited to the methods that directly detect hormones. The person skilled in the art will understand that detection of the hormones may be carried out by a number of methods well known in the art including, but not limited to, detection of the hormones directly by any available means—the means including, but not limited to, an immunoassay which may comprise a competitive immunoassay, an enzyme immunoassay, a ligand assay, an immunoradiomeric assay, a fluoroimmunoassay, an enzyme-linked immunosorbent assay or a radioimmunoassay.

The person skilled in the art will also understand that detection of hormone levels as contemplated in the embodiments of the present invention may be accomplished by methods that indirectly measure the levels of the hormones. Such methods include, but are not limited to, measurement of nucleic acid molecules encoding the hormones. These nucleic acids may include but are not limited to RNA or DNA.

In the context of the present invention the phrase “predicting the time of onset of labour” (and statements using similar language) means predicting, for example, whether labour will be preterm or not, and/or how many estimated days/weeks before the onset of labour whether it be preterm or full term. In this context, full term includes cases in which subjects have reached 37 to 40 weeks of pregnancy and also extends to situations in which the pregnancy has continued beyond 40 weeks. Length of pregnancy is determined according to any one of the commonly accepted means of determining length of pregnancy, including by ultrasound assessment, date of last menstrual period or any other relevant means.

### BRIEF DESCRIPTION OF THE FIGURES

FIG. 1 Smoothed median curves for singleton term and preterm delivery groups and twins.

FIG. 2. A P/E3 Ratio, B E3/E2 Ratio: Examples of estimated ratio trajectories for 3 pregnancies with spontaneous labour onset, shown for the 20 weeks prior to delivery. C Progesterone, D P/E2 Ratio, E P/E3 Ratio, F E3/E2 Ratio: 58 term pregnancies with spontaneous onset labour—estimated results at 18 weeks, 26 weeks and 4 weeks pre-delivery and measured results at labour.

FIG. 3. Measured levels and ratios for 172 samples in the 4 weeks pre-delivery, 58 samples at labor and 48 samples post-delivery. All these samples were from singleton, term pregnancies with spontaneous onset of labour.

FIG. 4. A. Progesterone, B. Estradiol, C. Estriol; Box and whiskers graphs of the PTD singleton group, Term singleton group and Twin pregnancy group with levels estimated at 26 weeks gestation by interpolation.

FIG. 5. Scatter plot of progesterone at 26 weeks against maternal weight at enrolment in 388 singleton pregnancies.

FIG. 6. A Progesterone, B Estradiol, C Estriol, D P/E2 Ratio: Examples of trajectories for 3 pregnancies with spontaneous labour onset. Ratios are shown for 20 weeks prior to delivery, together with ratios from measured samples. E Estradiol, F Estriol: 58 term pregnancies with spontaneous onset labour—estimated results at 18 weeks, 26 weeks and 4 weeks pre-delivery, measured results at labour.

FIG. 7. A Progesterone, C Estradiol, E Estriol: indicate the relationship of analyte levels measured from samples in the labor and post-delivery groups to corresponding predicted levels and prior samples for these 106 pregnancies. Note that these graphics are produced with a log scale to assist viewing because the ranges of hormonal values are extremely large.

B Progesterone, D Estradiol, F Estriol: indicate the relationship of analytes measured from samples at labour to corresponding predicted levels. Those points below the diagonal lines (of equality) show a decrease from predicted levels.

### DESCRIPTION OF THE INVENTION

Preferred embodiments of the invention will now be described with reference to the following non-limiting examples.

The inventors surprisingly found that ratios of particular hormones may be used to predict the timing of the onset of labour in pregnant subjects and, in particular, to predict the onset of preterm labour. Prediction of preterm labour would allow appropriate prophylactic treatment to prevent a preterm delivery and the associated risks to the neonate.

Samples for determining the levels/ratios of hormones may be in the form of blood, plasma, saliva, sputum, cervical or vagina smears or swabs. Detection of the hormones is preferably carried out in vitro. However, it will be understood that detection may be may be carried out in vivo.

### Example 1

Five hundred unselected pregnant women provided 2-9 plasma samples from 7 weeks of pregnancy to labour. Samples were assayed for progesterone, estradiol and estriol. Results were used to form trajectories for each analyte. Notably samples were taken between the hours of 9 am and 5 pm when no dramatic diurnal variation in P, E2 and E3 occurs (Keirse 1990).

### Study Design

The Human Ethics committee of the Hunter Area Health Service approved this study and all subjects provided written informed consent. A cohort of unselected subjects was recruited by research midwives at their first antenatal visit and followed to delivery at the John Hunter Hospital in Newcastle, Australia, during the period 2000-2004. Maternal blood samples were taken at approximately monthly intervals until and including sampling at the time of labour and just after delivery where possible. Visits to the ante-natal clinic were between 9 am and 5 pm. Gestational age was defined by an early ultrasound scan.

### Assay Methods

Blood was obtained by venepuncture, transferred to heparin tubes and centrifuged at 2000g at 4° C. for 15 minutes. Plasma was separated and kept at −20° C. until assayed. Samples for each subject were batched for assay.

Progresterone (P) and estradiol (E2) were measured, using the Bayer Corporation ADVIA Centaur assay (Bayer Corp., Tarrytown, N.Y., USA), a competitive immunoassay using direct chemiluminescent technology. For the P assay, sensitivity was 60 ng/mL and intra-assay CV 5.3%. For the E2 assay, sensitivity was 10 pg/mL and intra-assay CV 8.4%. Total estriol (E3) was measured using the Fluorescence Polarization Immunoassay (FPIA) technology and the Abbott TDxFLx analyser (Abbott Laboratories, Tex., USA). Sensitivity was 6.6 ng/mL and intra-assay CV 2.3%.

### Mathematical and Statistical Methods

Stata 9.2 software (StataCorp, College Station, Tex.) was used for curve-fitting and statistical analysis. Non-linear least squares estimation was used to fit individual curves for each woman and each analyte prior to the last four weeks of pregnancy.

Hypothesis tests of group medians or paired group medians were conducted using non-parametric statistical tests, as appropriate to the distribution of the data. A two-tailed significance level of 5% was used throughout. Means are reported with standard deviations (SD), medians are reported with either inter-quartile ranges (IQR) or bootstrapped 95% confidence intervals (CI) estimated by the bias-corrected and accelerated method (BCa)(Efton B and Tibshirani 1993).

### Results

### Subjects

Five hundred and fifty-seven women were recruited, of whom 57 were withdrawn due to incomplete attendance, formal withdrawals for a variety of reasons, 5 spontaneous abortions prior to 20 weeks and 2 terminations of pregnancy for fetal anomalies. Minimum study requirements were 2 blood samples taken and delivery and fetal outcome data available. Gestational length and gestational age at sample were based on early ultrasound scans except for 4 subjects in whom last menstrual period dating was used. The characteristics of the 500 subjects included are provided in Table 1.

TABLE 1
Maternal, Fetal and Pregnancy Characteristics

| Study Cohort | | Preterm Pregnancies | Term Pregnancies |
|---|---|---|---|
| (500 women) | | (n = 45) | (n = 455) |
| Plurality: | Singleton pregnancy | 35 | 452 |
| | Twin pregnancy | 9 | 3 |
| | Triplet pregnancy | 1 | — |
| Maternal age (years)* | | 28.7 ± 4.8 (21-41) | 27.9 ± 5.4 (16-47) |
| Primiparous | | 14 (31%) | 202 (44%) |
| Number of blood samples* | | 4.3 ± 1.3 (2-8) | 5.6 ± 1.1 (2-9) |
| Smoking-self reported: | at enrolment | 18 (40%) | 120 (26%) |
| | at delivery | 15 (33%) | 113 (25%) |
| Maternal morbidities: | Essential Hypertension | 1 | 2 |
| | Gestational Hypertension | 2 | 10 |
| | Pre-Eclampsia | 2 | 4 |
| | Gestational Diabetes | 1 | 1 |
| Labor onset: | Spontaneous | 24 | 307 |
| | Induced | 10 | 114 |
| | Caesarian | 11 | 34 |
| Fetal characteristics and | | | |
| outcomes | | Preterm (n = 56) | Term (n = 458) |
| Fetal sex: | Females | 29 | 223 |
| | Males | 27 | 235 |
| Neonatal outcomes: | Livebirth(s) | 48 (86%) | 456 (>99%) |
| | Stillbirth(s) | 7 | 2 |
| | Neonatal death(s) | 1 | — |

*Mean ± SD (minimum-maximum)

The women were predominately Caucasian (92%), with a small percentage of Aboriginal or Torres Strait Islander descent (3%) and others including Asians (5%). Maternal and fetal outcomes were much poorer for multiple gestations; preterm delivery rate was 7.2% in singleton and 77% in multiple gestations.

### Curve-Fitting

The following, further exclusion criteria were applied to subjects for curve-fitting: gestational length <26 weeks; <3 blood samples taken in total; <3 measurements for either P or E2 or E3 available before the last 4 weeks of pregnancy. 31 singleton, 2 twin and 1 triplet pregnancies were thus excluded. The remaining cohort of 466 subjects comprised a group of 456 women with singleton pregnancies (subgroups: 15 spontaneous onset preterm delivery (PTD: gestational length<37 weeks), 10 iatrogenic preterm delivery, 89 normal term, 313 other term, including induced and caesarean section delivery and 29 post-term), and a multiple gestation group of 10 women with twin pregnancies (4 spontaneous PTD, 4 iatrogenic PTD and 2 term pregnancies); a conservative definition of normal was used requiring spontaneous onset of labor, non-smoking and no pathology.

It was assumed that a single equation type could be used to curve-fit the samples for each analyte. Lower order equations were preferred. The samples in the last 4 weeks of pregnancy were excluded from curve-fitting to allow the detection of a late gestation change in trajectory. Further detail of curve-fitting is provided example 2 supplementary details of curve fitting. The following equations were selected:
loge progesterone=a+b*t1.5
(where t is gestational age in days, Coefficient of determination (R2)>0.85 for 95% of subjects, median for groups and subgroups range 0.96-0.97, overall median 0.97).
loge estradiol=a+b/(log t)
(R2>0.67 for 95% of subjects, median for groups and subgroups range
0.93-0.98, overall median 0.95).
loge estriol=a+b/✓t
(R2>0.74 for 95% of subjects, median for groups and subgroups range 0.89-0.97, overall median 0.95).

Estimated levels were calculated weekly for each subject and each analyte from the trajectory equations (using individual values of a and b) and the ratios P/E2, P/E3 and E3/E2 were calculated by division.

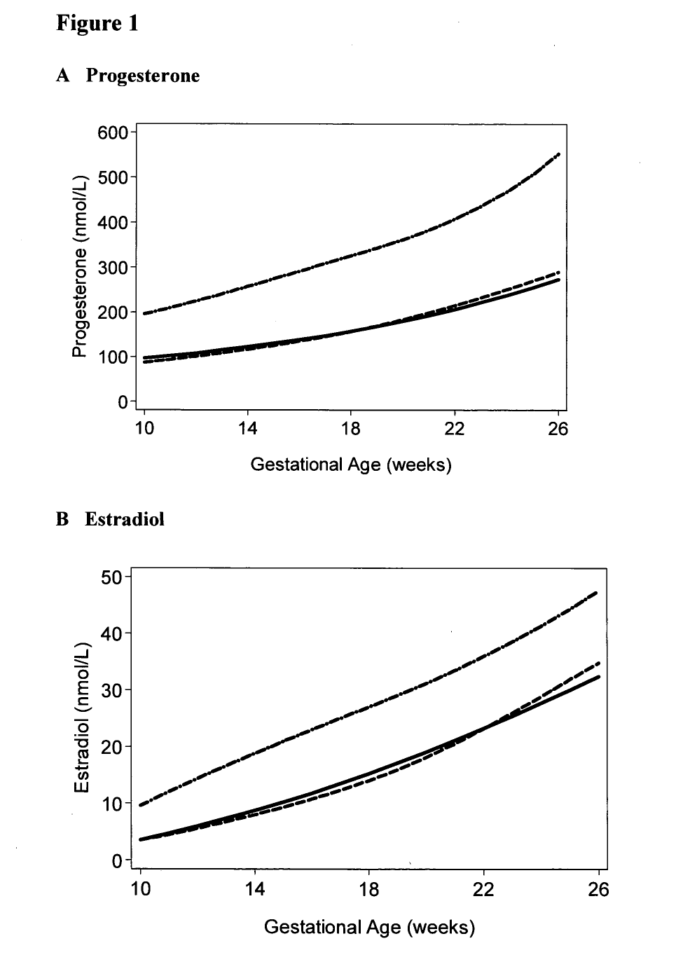

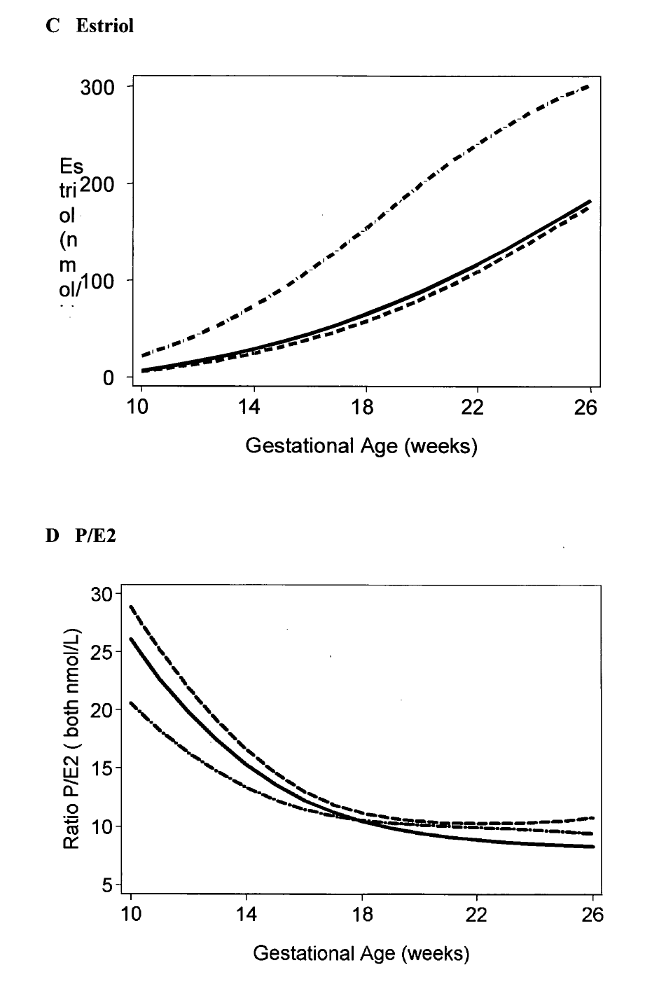

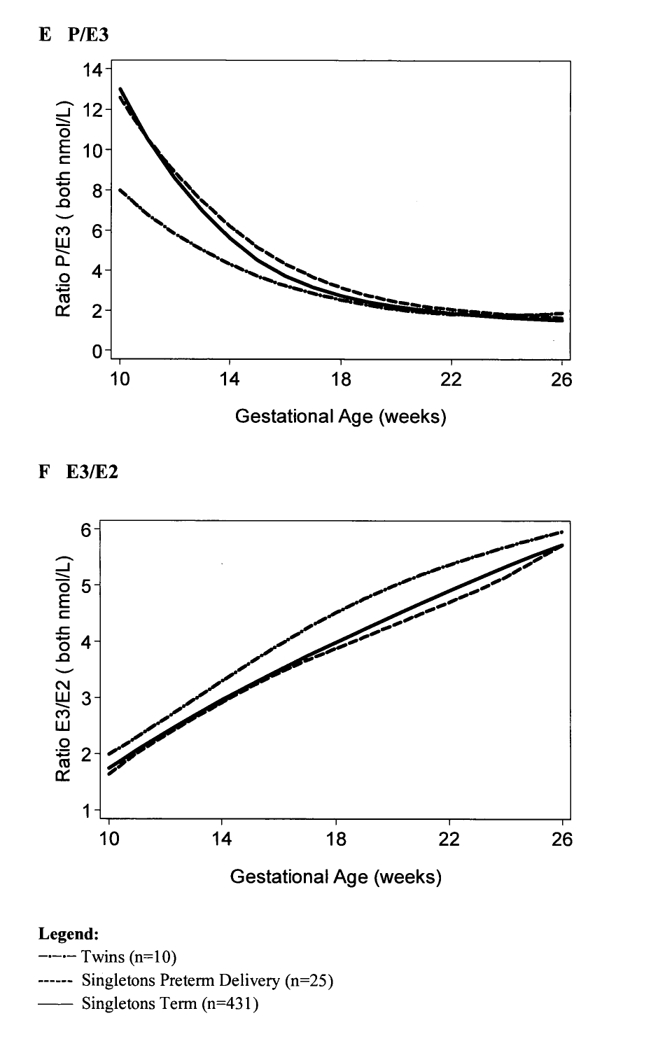

Smoothed, median curves for the twin group and preterm and term singleton groups are shown for P, E2, E3, P/E2, P/E3 and E3/E2 in FIG. 1.

Estimated median levels for P, E2 and E3 at 26 weeks in the twin pregnancy group (n=10) were considerably higher than the medians in the singleton group (n=456). Results are provided with medians and IQR in nmol/L: P, singletons 275 (235, 320), twins 504 (395, 722); E2, singletons 32.9 (24.0, 41.1), twins 45.6 (37.2, 91.9); E3, singletons 183 (143, 228), twins 351 (292, 601).

In 106 singleton, term pregnancies with spontaneous labor onset, additional blood samples were taken either in the 24 hours preceding delivery (labor group, n=58) or in the first 4 hours post-partum (post-delivery group, n=48). Additionally, 172 blood samples were taken in the last 4 weeks of pregnancy in 165 singleton, term pregnancies with spontaneous labor onset (last-4-weeks group).

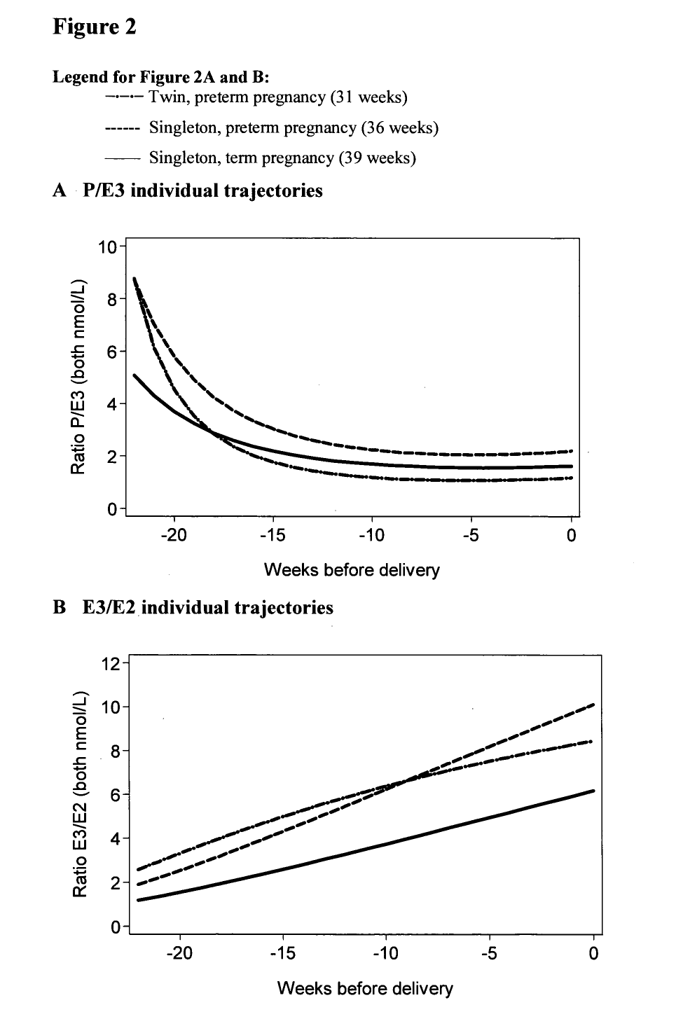

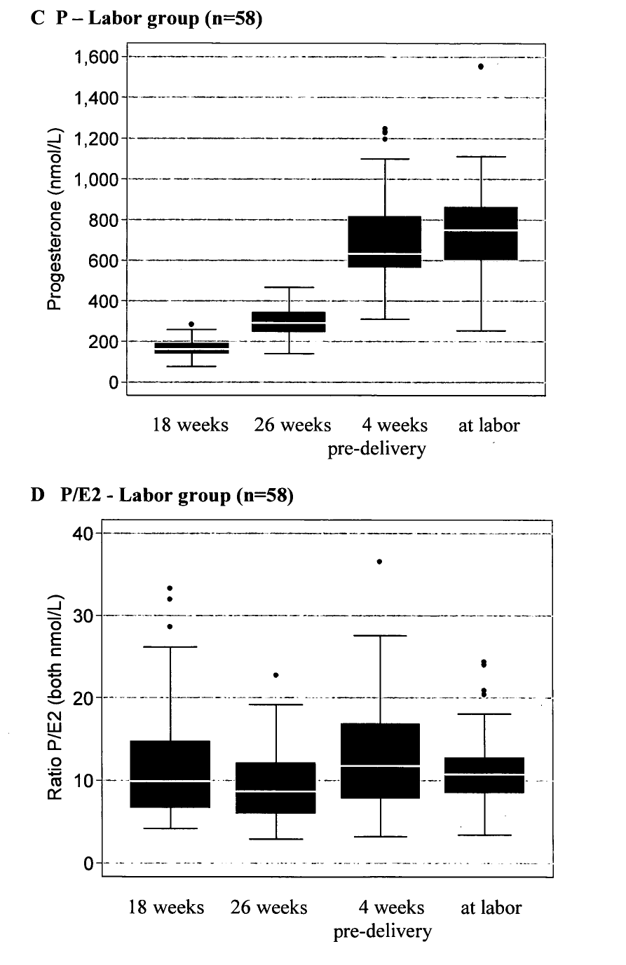

Examples of individual, estimated, trajectories for the ratios P/E3 and E3/E2 are shown in FIG. 2A-B for three pregnancies. These graphics show only the 20 weeks prior to delivery; note that some P/E3 ratios descend from very high levels in the early weeks of pregnancy. P, P/E2, P/E3 and E3/E2 results for the labor group (n=58) at four time-points are shown in box and whiskers plots in FIG. 2C-F; results are interpolated for the first 3 time-points and measured for the last.

Estimated levels for P, E2 and E3 interpolated at 26 weeks gestation for each pregnancy and the ratios P/E2, P/E3 and E3/E2 were compared in the singleton term (n=431) and preterm delivery groups (n=25) using Wilcoxon rank-sum group median tests. For the 58 pregnancies with a sample in the 24 hours prior to delivery (labor group), measured P, E2, E3, P/E2, P/E3 and E3/E2 results at labor were compared with estimated results 4 weeks prior using Wilcoxon matched-pairs signed-rank tests. In this group, samples at labor were also compared with measured penultimate samples; the mean time from the penultimate sample to the sample at labor was 24.2 days (SD 13.4). Results are provided in Table 2.

None of the hypothesis tests for the singleton preterm group versus the term group at 26 weeks showed a significant difference. For the labor group, all the paired hypothesis tests showed a significant difference, excepting the comparison of P levels and the comparison of P/E2 ratios. 53% of measured P levels at labor were lower than the previous measured level and the median paired difference between P level at labor and that at 4 weeks prior (48 nmol/L) was no different from zero, providing evidence that half of these trajectories had peaked before labor, however, the timing for this peak cannot be ascertained from these data.

Specifically, interpolated progesterone, estradiol and estriol median concentrations at 26 weeks gestation in singleton pregnancies with preterm deliveries (PTD) were not significantly different from those with term deliveries (P, p=0.63; E2, p=0.96; E3, p=0.29). In multiple pregnancies P, E2 and E3 concentrations were higher than in singletons. As pregnancy progressed the ratio of P to E2 in term singletons fell from 18;1 at 12 weeks to 11:1 at labour. P to E3 ratios fell from 7:1 at 12 weeks to 1:1 at labour, while E3 to E2 ratios rose from 2:1 at 12 weeks to 11:1 at labour.

The predicted levels at labour were compared with measured levels for P, E2 and E3 using matched-pair sign tests (binomial distribution). For P, 8 measured levels were above predicted and 50 below (p<0.001), for E2, 29 measured results were above and 29 below (p≈1.0) and for E3, 41 measured results were above predicted and 17 below (p=0.002).

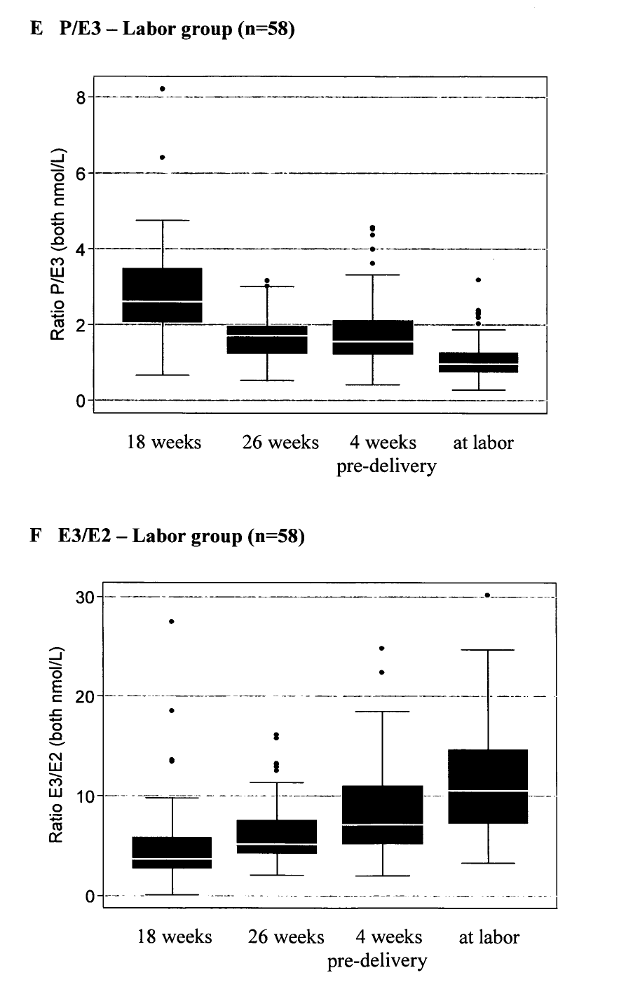

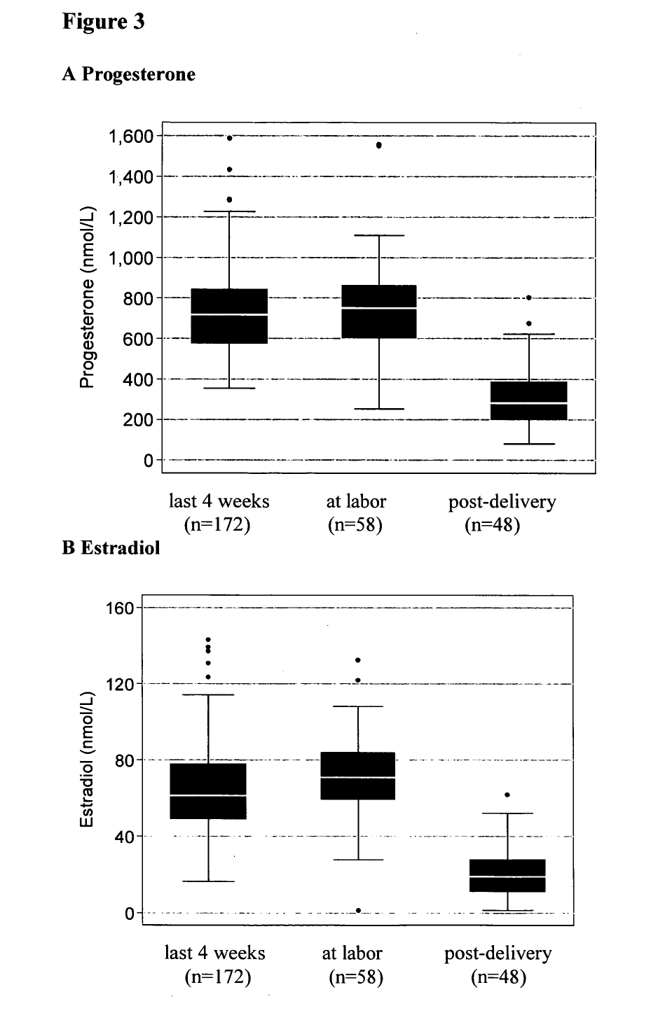

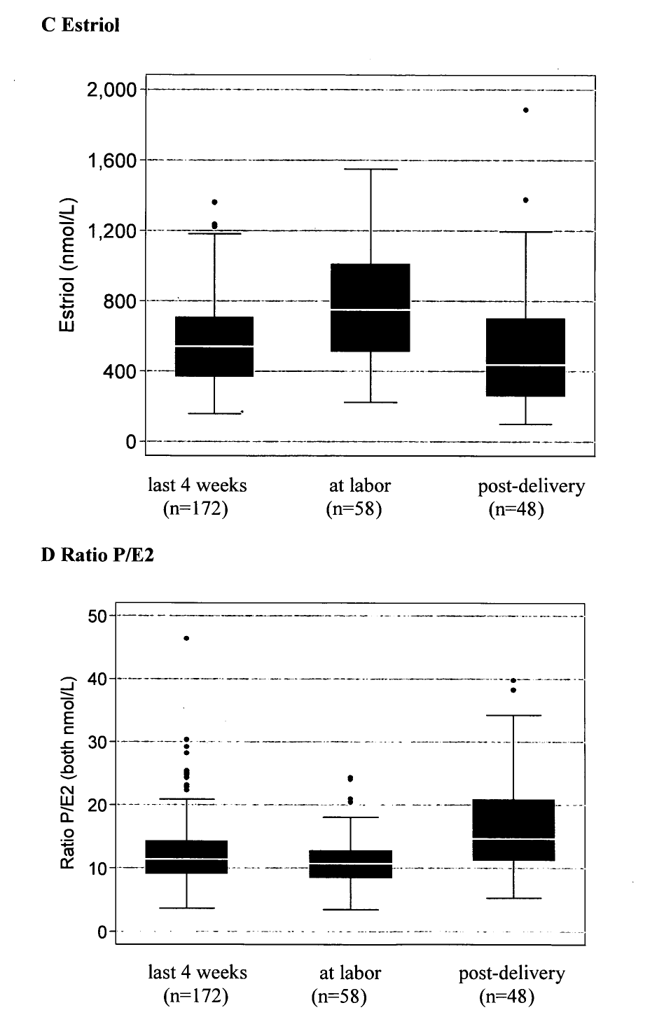

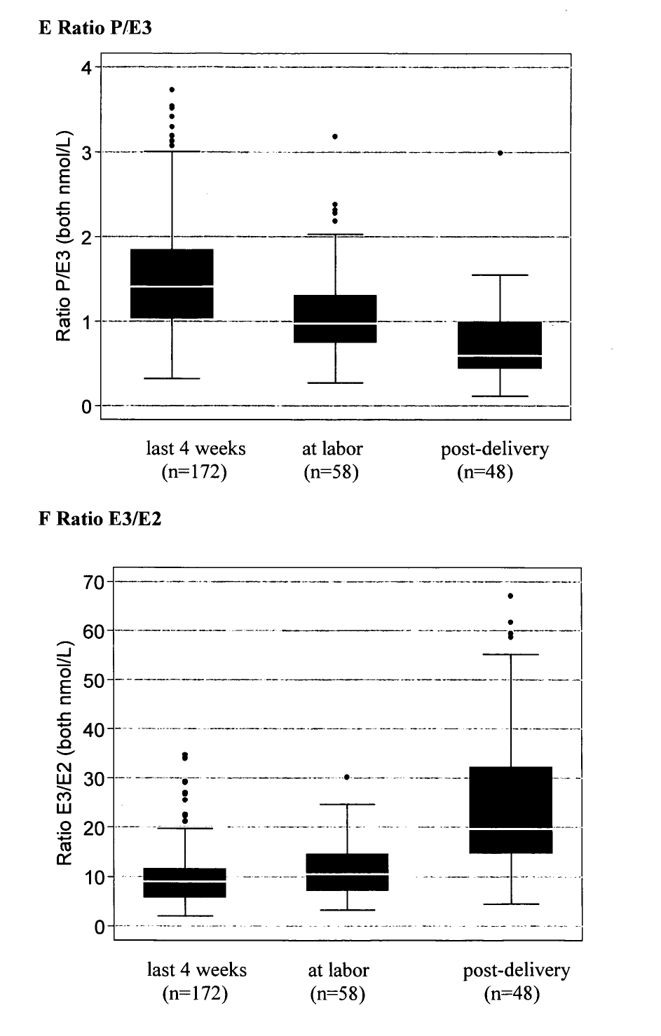

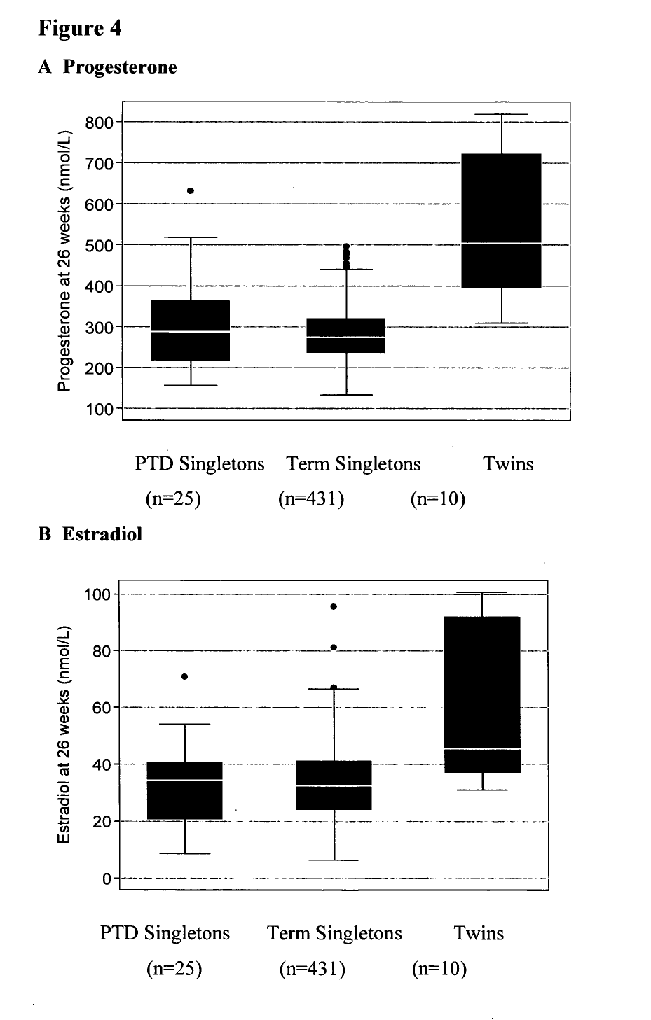

Box and whiskers graphs for measured, late-pregnancy samples are provided in FIG. 3.

### Example 2

### Supplementary Curve Fitting Details Follow.

For P, E2 and E3, a test group of 6 normal subjects with 7 or more samples was used to examine the trajectory fit with various curve types and choose several candidate equations, which were then fitted to the whole group and a final choice of curve-type selected. Given that intra-assay CVs are similar percentages at low, medium and high levels, log-transforming and using a weighting of I provided a reasonable approach for regression considering that the range of levels for each pregnancy was extremely wide (from <100 to several thousand units in some cases). Initially, a range of equations of order 1 (i.e. degree I using a fractional polynomial approach Royston P. 1994; and Royston et al 1999) and a quadratic equation were tried; had none of these proved satisfactory because of the trajectory shapes, further equations of degree 2 would have been used. Log-transformed data were curve-fitted for each analyte and for each subject by non-linear least squares regression. The set of possible equation types which fitted the test group well was then fitted to the data for each of the 466 subjects, using an automated process and generating individual coefficients a, b and c (quadratic equation only) for each equation for each subject, Individual trajectories were compared visually against sample levels for each analyte in the test group and for other subjects if the R2 fit parameter was low.

Several criteria were used for the final choice of equation for each analyte:
approximate normality and homoscedasticity in combined residuals
consideration of median R2, both in the whole group and sub-groups. The chosen equation should not fit differentially in singleton subgroups and if the twin pregnancies had trajectories quite different from singletons, it may have been necessary to use a different equation type for twins. A higher overall median R2 was preferred.
a curve-type of lower order was preferred if the fit was comparable to an equation of higher order.

The resultant equations for P, E2 and E3 were monotonically increasing for all subjects:
progesterone=exp(a+b*t1.5)
where t is gestational age in days
estradiol=exp(a+b/(log t))
estriol=exp(a+b/✓t)

In order to interrogate the wide variation in progesterone levels, linear regression was used to assess the association of progesterone levels estimated at 26 weeks gestation with maternal and fetal factors (which may be causal or confounding) using the following continuous and dichotomous variables in 388 singleton pregnancies (367 term and 21 PTD pregnancies with all data items available): maternal age, over 35 years (Y/N), parity, primiparous (Y/N), maternal weight at enrolment, smoking prior to enrolment (Y/N), fetal sex. Those variables with p values lower than 0.25 in simple linear regression were included in multiple regression in backwards stepwise mode. However, where variables were highly correlated (such as parity and primiparous), only the variable with the lowest p value was included in multiple regression.

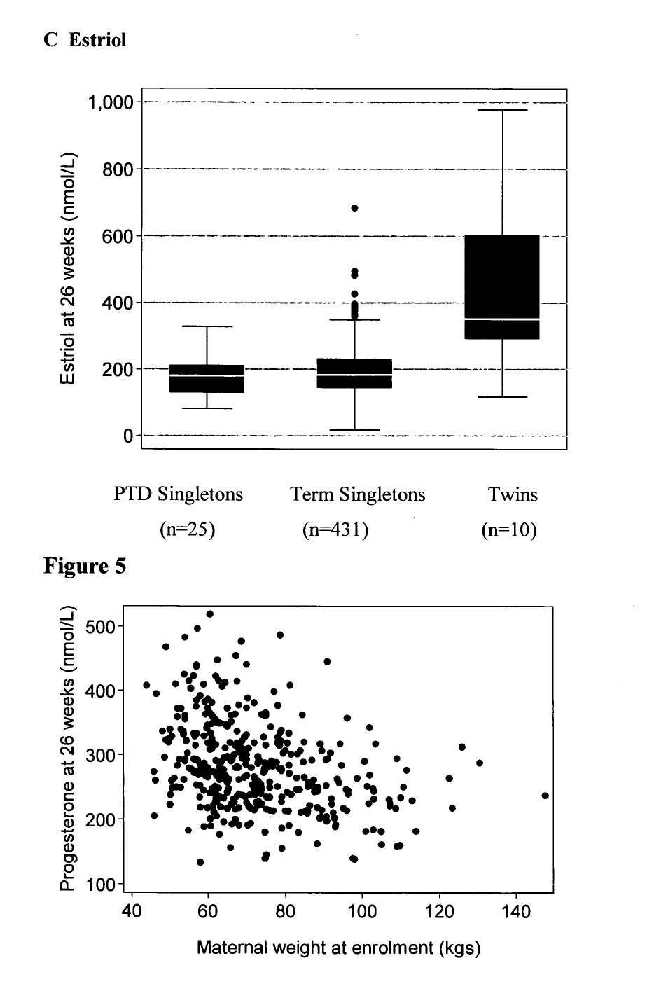

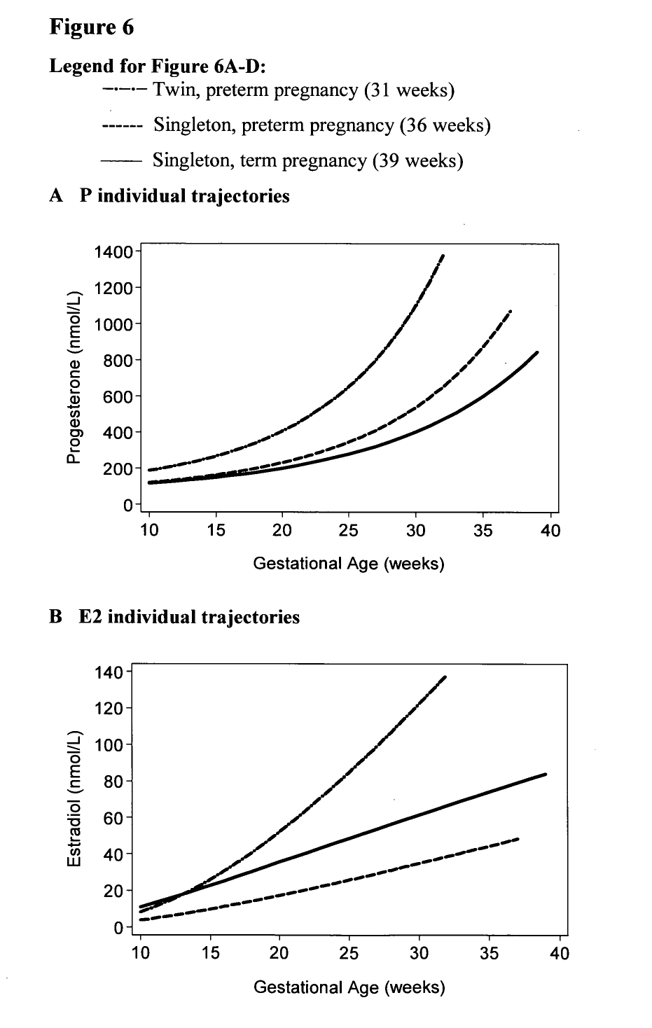

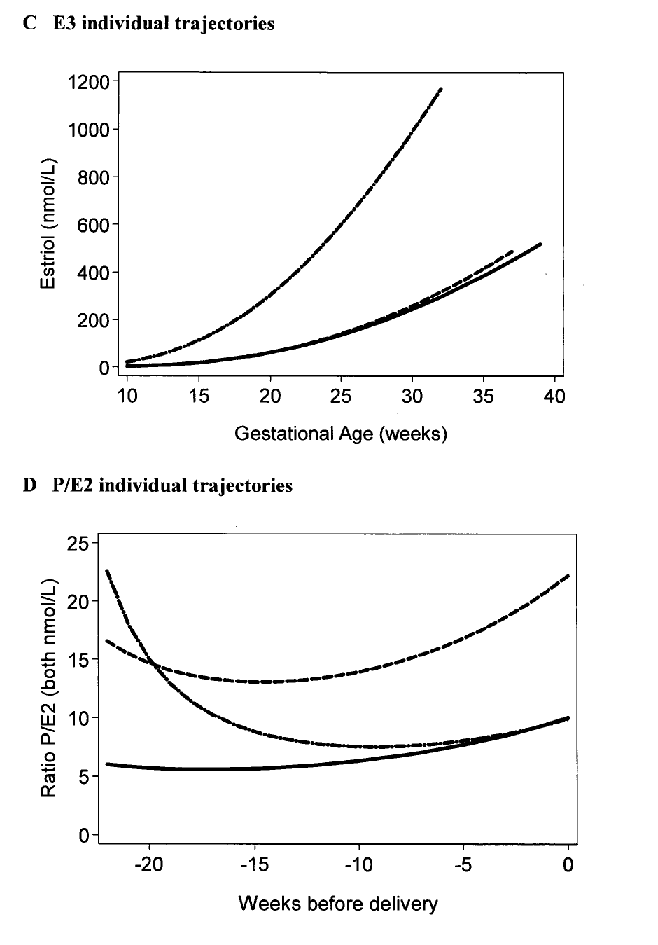

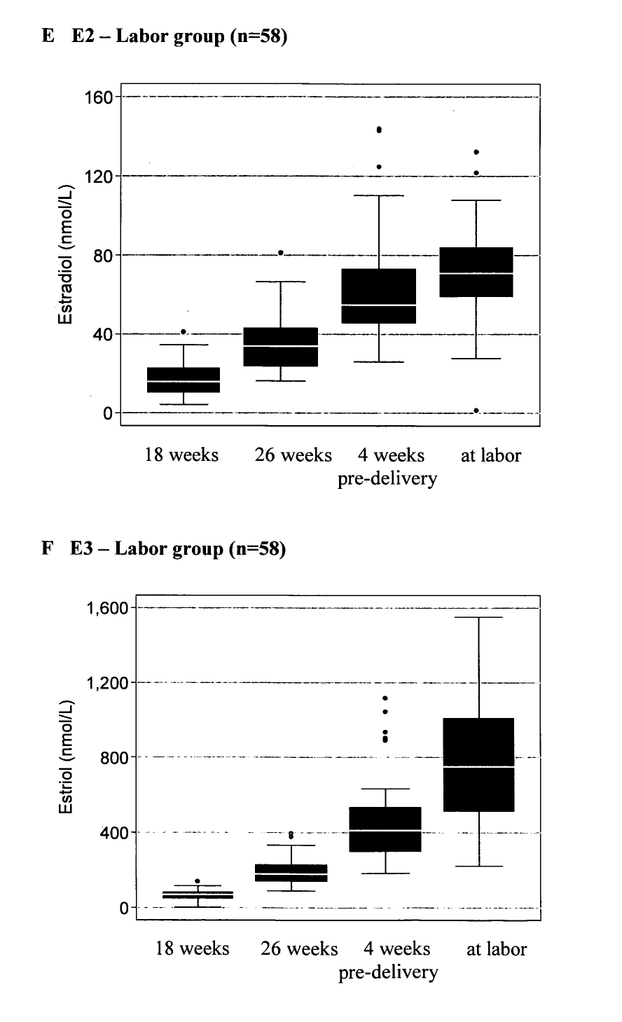

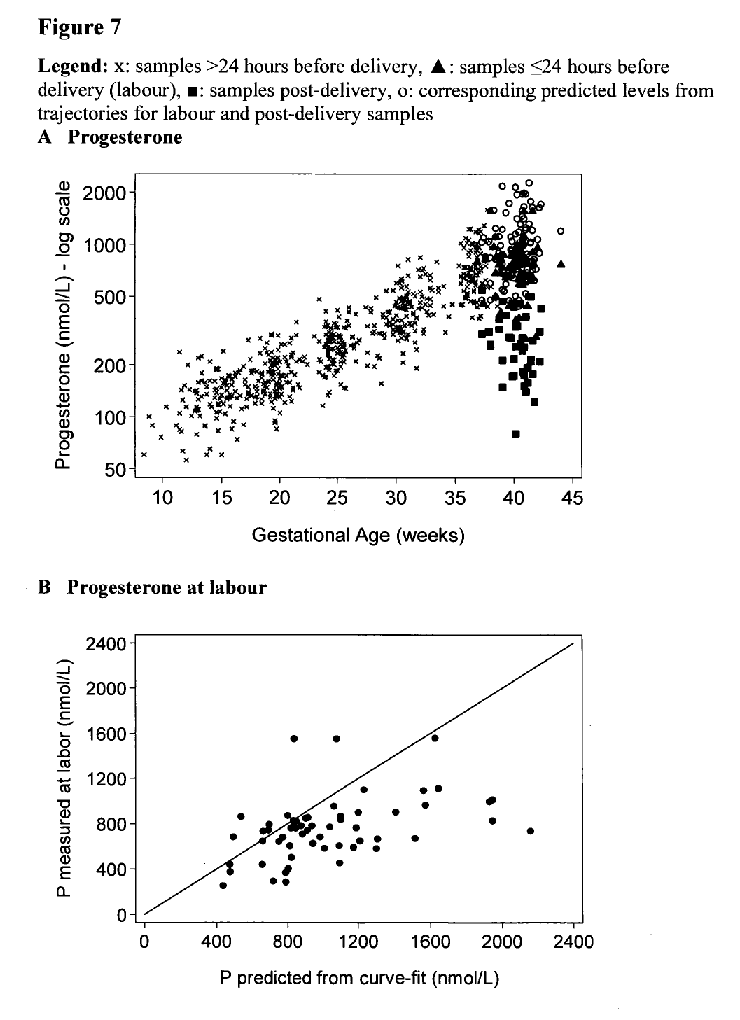

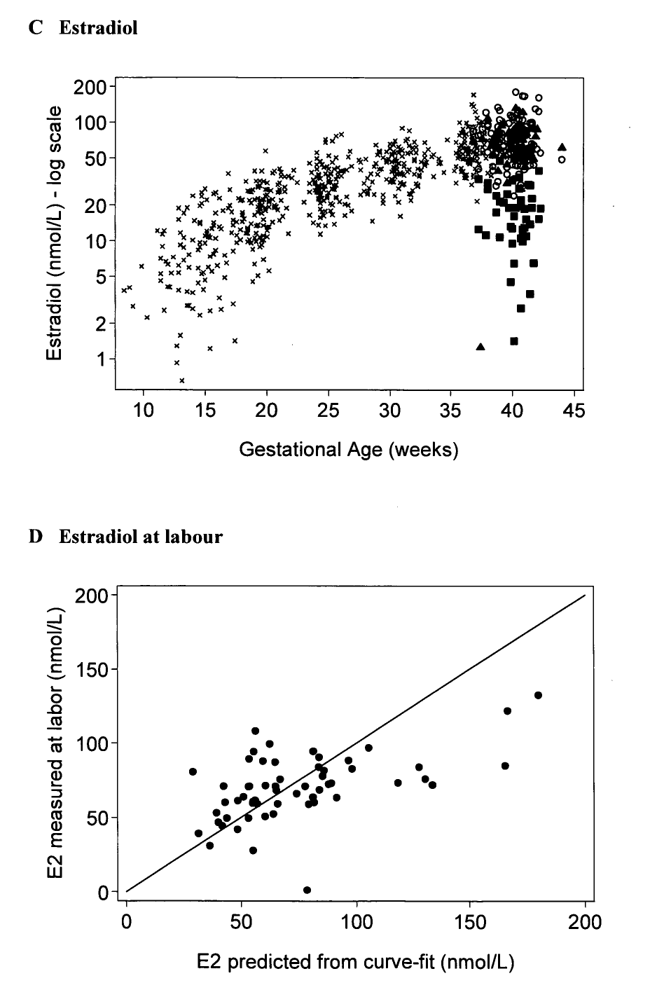

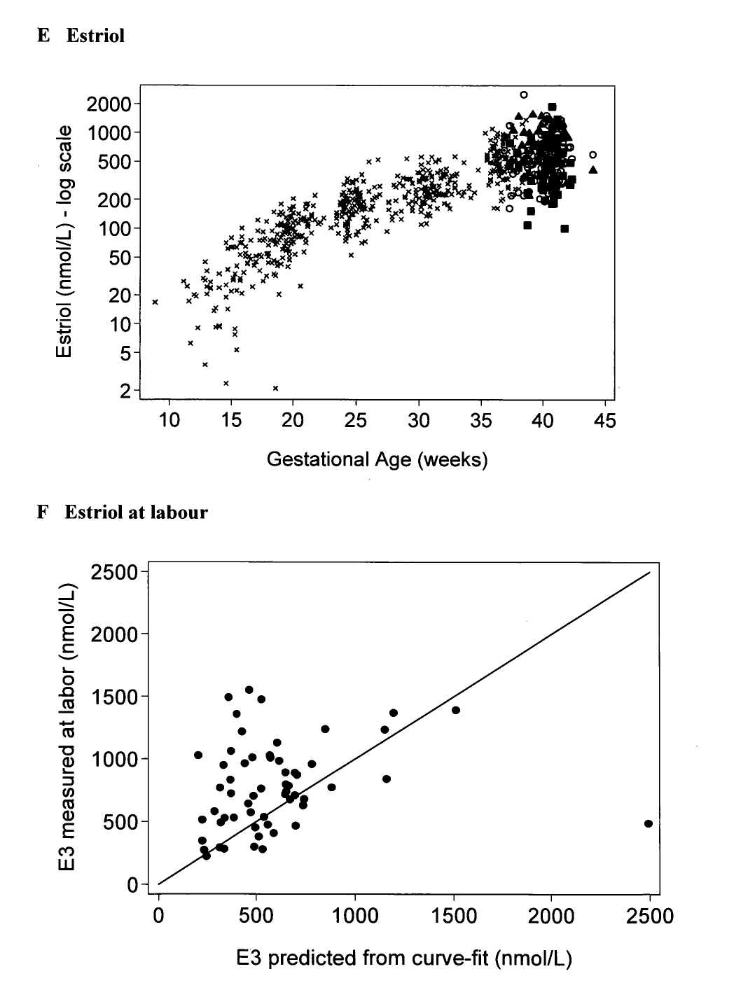

The association of progesterone estimated at 26 weeks gestation with maternal and fetal predictor variables was assessed initially using scatterplots; maternal weight showed an inverse (curved) relationship with progesterone and higher progesterone levels only occurred at lower weights (weight range 44-148 kgs; Term mean weight 72.2 kg (SD 16.2), PTD mean weight 71.2 kg (SD 20.0))(FIG. 5). Weight was transformed to the inverse form for use in regression. Results of simple linear regression for progesterone against each predictor variable were as follows, including p values and t statistics: age, p=0.94, t=0.08; over 35 years, p=0.89, t=−0.14; parity, p<0.001, t=−3.84; primiparous, p<0.001, t=3.91; inverse weight, p<0.001, t=7.56; male sex, p=0.001, t=3.45; smoking, p=0.021, t=−2.32.

Inverse weight and dichotomous variables primiparous, fetal sex and smoking were included in the multiple linear regression; these four variables were simultaneously significantly associated with progesterone level at 26 weeks. P-values, coefficients and 95% CIs were as follows: inverse weight, p<0.001, 7967 (5879, 10054); primiparous, p=0.003, 18.7 (6.2, 31.1); male sex, p<0.001, 22.6 (10.4, 34.9); smoking, p=0,002, −22.2 (−35.9, −8.6); constant 154 (123, 185). 20% of the variance in progesterone levels is explained by the model (R2=0.20), indicating higher progesterone levels (on average) for those women having their first baby, non-smokers and for males and decreasing levels with increasing maternal weight (after adjustment for the other variables in the model). There was significant interaction between inverse weight and smoking (p=0.03, coefficient −4877 (−9356, −398)); with the interaction term included in the model, R2 was slightly increased to 0.21 and the coefficients of the other variables were changed. The assumptions of linear regression were met but the residuals were fairly large, probably due to these variables only explaining a modest proportion of the variance.

TABLE 2
Results of hypothesis tests and comparisons of samples at labour with penultimate samples.
Comparison of measured samples at labour with respective estimates 4 weeks prior to delivery,
measured penultimate samples and predicted results at labour (n = 58)

| | | | | Number of | |
|---|---|---|---|---|---|
| | | | | samples at labour | |
| | Estimated Median | | Paired | decreased from | |
| | 4 weeks prior to | Measured Median | test: | penultimate | Predicted Median |
| | delivery, (95% Cl) | at labour (95% Cl) | P value | (with %) | at labour (95% Cl) |
| P (nmol/L) | 632 (596, 746) | 751 (682, 801) | 0.14 | 31 (53%) | 915 (835, 1073) |
| E2 (nmol/L) | 54.93 (50.43, 67.45) | 70.86 (63.39, 74.38) | 0.003 | 21 (36%) | 64.87 (57.08, 81.08) |
| E3 (nmol/L) | 412 (380, 475) | 750 (658, 854) | <0.001 | 15 (26%) | 528 (466, 605) |
| P/E2 ratio | 11.78 (9.57, 14.29) | 10.79 (9.76, 12.07) | 0.07 | 39 (67%) | 13.98 (11.33, 17.46) |
| P/E3 ratio | 1.55 (1.46, 1.76) | 0.98 (0.88, 1.11) | <0.001 | 47 (81%) | 1.75 (1.58, 2.07) |
| E3/E2 ratio | 7.04 (5.71, 8.24) | 10.59 (8.65, 13.4) | <0.001 | 19 (33%) | 7.76 (6.07, 9.39) |

Comparison of singleton term and PTD groups with results estimated at 26 weeks gestation

| | Term group (n = 431) | PTD group (n = 25) | |
|---|---|---|---|
| | Median (95% Cl) | Median (95% Cl) | P value |
| P (nmol/L) | 274 (268, 281) | 288 (231, 363) | 0.63 |
| E2 (nmol/L) | 32.56 (31.57, 34.66) | 34.3 (27.64, 40.04) | 0.96 |
| E3 (nmol/L) | 183 (179, 191) | 180 (135, 204) | 0.29 |
| P/E2 ratio | 8.37 (8.1, 8.9) | 10.94 (7.85, 13.37) | 0.26 |
| P/E3 ratio | 1.53 (1.47, 1.61) | 1.64 (1.31, 1.95) | 0.23 |
| E3/E2 ratio | 5.73 (5.48, 6.07) | 5.67 (4.81, 7.86) | 0.74 |

Although the invention has been described with reference to specific examples, it will be appreciated by those skilled in the art that the invention may be embodied in many other forms, in keeping with the broad principles and the spirit of the invention described herein.

### REFERENCES

Boroditsky R S, Reyes F I, Winter J S, Faiman C. Maternal serum estrogen and progesterone concentrations preceding normal labor. Obstet Gynecol 1978;51:686-91.
Csapo A. Progesterone block. Am J Anat 1956;98:273-91.
da Fonseca E B, Bittar R E, Carvalho M H, Zugaib M. Prophylactic administration of progesterone by vaginal suppository to reduce the incidence of spontaneous preterm birth in women at increased risk: a randomized placebo-controlled double-blind study. Am J Obstet Gynecol 2003;188:419-24
Efron B, Tibshirani R. An introduction to the bootstrap. New York: Chapman & Hall; 1993
Gibb W, The role of prostaglandins in human parturition.1998 Ann Med 30 235-241.
Goffinet F, Rozenberg P, Kayem G, Perdu M, Philippe H J, Nisand I, The value of intravaginal ultrasonography of the cervix uteri for evaluation of the risk of premature lab. J. Gynecol. Obstet. Biol. Reprod (Paris) 1997; 26:623-9
Iams J. Prevention of preterm birth. N Engl J Med 1998; 338: 54-6
Keirse M J. Progestogen administration in pregnancy may prevent preterm delivery. Br J Obstet Gynaecol 1990;97:149-54
Meis P J, Klebanoff M, Thom E, et al. Prevention of recurrent preterm delivery by 17 alpha-hydroxyprogesterone caproate. N Engl J Med 2003;348:2379-85
Mesiano S. Roles of estrogen and progesterone in human parturition. Front Horm Res 2001; 27:86-104.
Royston P. Regression using fractional polynomials of continuous covariates: parsimonious parametric modelling. Appl Stat 1994;43:429-67.
Royston P, Ambler G, Sauerbrei W. The use of fractional polynomials to model continuous risk variables in epidemiology. Int J Epidemiol 1999;28:964-74
Tulchinsky D, Hobel C J, Yeager E, Marshall J R. Plasma estrone, estradiol, estriol, progesterone, and 17-hydroxyprogesterone in human pregnancy. I. Normal pregnancy. Am J Obstet Gynecol 1972;112:1095-100.
U.S. Pat. No. 5,650,394 Terao T, Kanayama N, Casal D, (filed 4 Nov. 1993)
U.S. Pat. No. 5,516,702 Seyei A. E, and Casal D. C, (filed 29 Jun. 1994)

1-59. (canceled)

60. A method of predicting an increased risk of onset of labour in a pregnant human subject, wherein the method comprises the steps of:
(a) obtaining at least one sample from the subject;
(b) determining a ratio of estriol level to estradiol level in the at least one sample; and
(c) predicting the risk of onset of labour,
wherein a ratio of the estriol level to the estradiol level greater than about 8:1 indicates the increased risk of onset of labour.

61. The method of claim 60, wherein the subject is preterm.

62. The method of claim 60, wherein the subject is full term.

63. The method of claim 60, wherein the at least one sample is selected from the group consisting of blood, plasma, saliva, cervical secretions, and vaginal secretions.

64. The method of claim 63, wherein the at least one sample is a blood sample.

65. The method of claim 60, wherein the level of estriol or estradiol is determined by an assay selected from the group consisting of a competitive immunoassay, an enzyme immunoassay, a ligand assay, an immunoradiomeric assay, a fluoroimmunoassay, an enzyme-linked immunosorbent assay, and a radioimmunoassay.

66. The method of claim 60, wherein the at least one sample is obtained from the subject after week 26 of gestation.

67. The method of claim 60, wherein the at least one sample is obtained from the subject in the time period between week 26 to week 37 of gestation.

68. The method of claim 60, wherein in step (a) more than one sample is obtained from the subject.

69. The method of claim 68, wherein a trajectory of the ratios determined for the more than one sample is estimated.

70. The method of claim 60, wherein the risk is expressed as a predicted time to the onset of labour.

71. The method of claim 70, wherein the time to onset of labor is predicted to be within 77 days or less from the time the at least one sample was obtained.

72. The method of claim 71, wherein the time to onset of labor is predicted to be within 7 days or less from the time the at least one sample was obtained.

73. The method of claim 72, wherein the time to onset of labor is predicted to be within 2 days or less from the time the at least one sample was obtained.

74. The method of claim 73, wherein the time to onset of labor is predicted to be within 1 day from the time the at least one sample was obtained.
